(19) United States
(12) Patent Application Publication
(10) Pub. No.: US 2010/0086571 A1
(43) Pub. Date: Apr. 8, 2010

(54) METHODS TO DECREASE THE RISK OF METABOLIC SYNDROME POST IMMUNIZATION

(76) Inventor: John Barthelow Classen, Baltimore, MD (US)

Correspondence Address: BROWDY AND NEIMARK, P.L.L.C.;624 NINTH STREET, NW, SUITE 300, WASHINGTON, DC, 20001-5303, US

(21) Appl. No.: 12/444,232

(22) PCT Filed: Oct. 5, 2007
(86) PCT No.: PCT/US07/80550

§ 371 Date: Nov. 25, 2009

Related U.S. Application Data
(60) Provisional application No. 60/849,448, filed on Oct. 5, 2006.

(51) Int. Cl. A61K 39/00; G06Q 90/00

(52) U.S. Cl. 424/277.1; 424/184.1; 705/500

(57) ABSTRACT

This invention contemplates (I) assessing the risk of metabolic syndrome and or one of its component diseases/disorders which is associated with an immunization schedule against one or more infectious diseases, or one or more immunogens which induce protective immunity against infectious diseases, (II) screening one or more potential recipients and identifying at least one human subject who would be expected to be immunized safely with said one or more immunogens or said immunization schedule reflective of the analysis from (I), and (III) immunizing said human against said one or more infectious diseases.

This application claims the benefit, pursuant to 35 USC 119(e), of prior U.S. provisional application 60/849,448, filed Oct. 5, 2006, hereby incorporated by reference in its entirety. Likewise, outside the U.S., it claims the benefit of said application under the Paris Convention.

### BACKGROUND OF THE INVENTION

1. Field of the Invention

The present invention involves the fields of immunology and medicine, and more particularly relates to immunization methods, and compositions used therewith, for immunizing mammals, such as human infants and children, against infections while avoiding undesired increases in metabolic syndrome and components including obesity, hypertension, hyperlipidemia, low HDL cholesterol, insulin resistance, and microalbuminurea.

2. Related Background Art

Vaccines have been shown to cause a number of autoimmune diseases including diabetes (1-4). The inventor has previously been issued a number of patents on technology to prevent “chronic immune mediated disorders”. These diseases comprise autoimmune diseases, allergies and immune mediated cancers (U.S. Pat. Nos. 5,723,283; 5,728,385; 6,420,139; 6,584,472; 6,638,739; 7,008,790). Several additional patent applications have been filed by this inventor including (US application Ser. No. 10/602,772; PCT/US 2005/011386 or WO 2005/097188 A3). In these documents a comprehensive list of immunological diseases that had been linked or thought to be linked to immunization were cited. However in none of these was metabolic syndrome and or its components including obesity, hypertension, hyperlipidemia, low HDL cholesterol, insulin resistance, or microalbuminurea considered chronic immune mediated disorders.

There is an epidemic of several diseases in human children and adults including hypertension, obesity, hyperlipidemia, low HDL cholesterol, microalbuminurea, and insulin resistance (5, 6). This syndrome has been labeled metabolic syndrome (7) and is closely associated with type II diabetes (8) and other health problems including death (9). The etiology is unknown but many have blamed poor diet (10) and lack of exercise. Diet and exercise have been touted as the cure for metabolic syndrome but have not been very effective (11) and have not stopped the epidemic of disease to date. The diet exercise theory does not explain the obesity epidemic in children under 6 month of age who don't drink many sodas, don't eat a lot of fried potatoes and have never been very active. Recent data from a Massachusetts HMO shows a 73% increase in overweight infants under 6 months of age from 1980 to 2001 (12).

While type II diabetes secondary to partial destruction of islet cells has been linked to immunization, neither obesity, hypertension, hyperlipidemia, low HDL cholesterol, insulin resistance, microalbuminurea nor metabolic syndrome have been linked before. To date no one has linked metabolic syndrome to vaccination. Tsai (13) studied the effect influenza vaccine on inflammation and lipid profile for just 7 days after immunization and concluded influenza vaccination causes “transient changes” in selected markers of inflammation and lipids. The study only involved 22 healthy patients. Numerous groups have also published papers showing that vaccines transiently increase inflammation (14-18). These studies followed a small number of patients (generally 20 or less) for a short period of time (generally a few days to 4 weeks) and did not find any evidence that vaccines cause chronic inflammatory conditions nor did they look for an effect on metabolic syndrome or its components. Several papers have shown that immunization can alter cortisol release at least in the short term (19-24) but again never linked this to metabolic syndrome or its components. Researchers in France have linked aluminum adjuvants in vaccines to an inflammatory condition called myofascititis (25, 26) but not to metabolic syndrome or its components. Again the study size was small and limited to case series. Immunization has been linked to transient increased cortisol production (27) but not to chronic Cushing's Disease like syndrome resembling metabolic syndrome. Fagliolo (28) showed an increased production of cytokines by mononuclear cells in the elderly and suggested it may result in chronic disease including atherosclerosis. However the author did not mention metabolic syndrome or that vaccines could be causing this.

Wang (29) studied the effect of influenza vaccine on hospitalization for numerous causes including diabetes, cancer. Wang was not implying the influenza vaccine caused cancer, diabetes, strokes, however he was indicating that it prevented patients with cancer, diabetes, cardiac/vascular disease etc. from being hospitalized. Since the vaccinated group had fewer hospitalizations the data would not lead anyone to believe that immunization caused metabolic syndrome.

Several groups have attempted to develop vaccines to modulate hypertension, hypercholesterolemia, and other components of metabolic syndrome by blocking hormones, lipids, receptors which are involved or modulate these conditions. For example Brown (30) developed a vaccine to prevent hypertension by blocking angiotensin. These type of specific vaccines designed to induce an therapeutic autoimmune like effect are fundamentally different that the current invention which pertains to decreasing an undesired and previously unknown adverse event of immunization, metabolic syndrome and its components. On the other hand it has been published (31) that a specific vaccine, BCG, induces an autoimmune disease to HSP, which causes atherosclerosis. Atherosclerosis is not a component of metabolic syndrome and this mechanism of inducing a specific autoimmune disease is not part of the current invention.

In conclusion, it has heretofore not been clearly shown or recognized that vaccines are associated with an increased the risk of developing metabolic syndrome or one of its components. Furthermore no one has indicated that because of an effect of routine immunization on metabolic syndrome or its components that changes should be made in immunization practices, selection of individuals to immunize, choosing of schedules, choosing of vaccines, and formulations of vaccines.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

### SUMMARY OF THE INVENTION

The present invention relates to the discovery that vaccines and immune modulators are associated with an increased rate of metabolic syndrome including its components hypertension, hyperlipidemia, low HDL cholesterol, insulin resistance, microalbuminurea and obesity. Furthermore the effect is dose/schedule dependent. The more vaccines/doses one receives the greater the risk.

While not being limited to a specific mechanism of action, immunization can cause an increase in metabolic syndrome by stimulating the immune system and increasing glucocorticoids (i.e. cortisol) from the adrenal glands. Cortisol is known to cause hypertension through its affect on salt retention in the kidney, as well as increasing hyperlipidemia and obesity.

Without intending to be bound by any theory, administration of immunogens can cause the release of lymphokines that causes cortisol release and a Cushingoid syndrome. The immunization may act in several ways including:

A. Continued release of cortisol or increased cellular uptake of cortisol because of chronic stimulation from chronically activated immune cells such as macrophages.
B. Resetting of the pituitary or adrenal gland leading to chronic increased production of cortisol.

Vaccines may be made safer by testing specific vaccine formulations and immunization schedules for their ability to affect these conditions. Vaccine manufacturers can further provide warnings to allow the patient, medical provider or other decision maker to consider the risk prior to immunization. The risk of vaccine-induced disorders is dependent on the immunization schedule, or using a decreased dosage of vaccine or fewer doses. Safer vaccine kits can be provided by including the warnings on the package inserts.

### DETAILED DESCRIPTION OF THE PREFERRED EMBODIMENTS OF THE INVENTION

It has now been discovered that immunization is associated with an increased risk of metabolic syndrome and its components. Furthermore the effect is dose/schedule dependent. The more vaccines/doses one receives the greater the risk. Metabolic syndrome is defined herein to comprise any use of the term or any accepted definition encompassing a combination of metabolic parameters (its components). As defined herein “its components” is defined as an alteration consistent or congruent with the alterations described in a paper by Ford (7) including obesity, abdominal obesity, hypertriglyceridemia and or hyperlipidemia, low HDL, high blood pressure, insulin resistance and microalbuminurea. High fasting glucose, caused by decreased insulin secretion/diabetes has previously been associated with immunization and by the definition herein is not intended to be claimed by itself as “one of its components” included as a sole component in the claim language. Insulin intolerance however is a newly discovered association with immunization and is intended to be claimed as a sole component of metabolic syndrome.

Preferably, the above mentioned risk information is included in the labeling for a kit with the vaccine. However, it is anticipated that some vaccine manufacturers or marketer/distributor/reseller may help inform physicians/healthcare providers/consumers by using other methods of disseminating this information. For example, and without limitation, a vaccine manufacturer or marketer/distributor/reseller may inform potential consumers by providing or assisting the dissemination of information electronically (e.g., over the Internet), in print (e.g., academic publications, catalogues, advertisements), and orally (e.g., in presentations at conventions and at hospital or HMO staff meetings or retreats). The manufacturer or marketer/distributor/reseller can disseminate the information directly (e.g., by placing advertisements, making presentations, and distributing publications) or it can act indirectly to disseminate the information (e.g., by subsidizing a person writing a publication, subsidizing a journal where the publication is printed, or subsidizing a meeting where the information is being presented).

It is the intention of the inventor to claim safer methods of marketing a vaccine which comprise providing the newly discovered information as well as the vaccine, whether or not simultaneously, and regardless of the means used to disseminate the information.

There are many ways the discoveries can be incorporated into safer methods of immunization.

The physician can screen patients for metabolic syndrome or one of its components and avoid immunizing a patient if the patient has metabolic syndrome or one of its components or is borderline for developing a component such as borderline hypertension for example.

The physician can screen patients for a family history or medical history of metabolic syndrome, hypertension, obesity, hypertriglyceridemia and or hyperlipidemia, low HDL cholesterol, insulin resistance, microalbuminurea and avoid immunizing the patients with a family history or medical history.

The physician can screen those with a medical history or family history of metabolic syndrome, hypertension, obesity, hypertriglyceridemia and or hyperlipidemia, low HDL cholesterol, insulin resistance, microalbuminurea and immunize only those with an elevated risk of developing a serious infection which is prevented by a vaccine.

The physician can measure evidence of immunological changes after immunization and only provide booster doses or additional vaccines if the patient seems to tolerate the vaccine.

The physician can decrease the number of doses or amount of vaccine given after 42 days of life.

The physician can screen women for pregnancy and avoid giving vaccines to pregnant women, especially if there is an increased risk of metabolic syndrome. The physician can warn women about the risk of the child developing metabolic syndrome or its components if the woman is considering being immunized when pregnant.

The physician may administer vaccine formulations less likely to induce chronic inflammation and or metabolic syndrome or one of its components. Such a formulation can include a vaccine lacking an aluminum adjuvant or a poorly metabolized antigen such as a complex polysaccharide.

A vaccine (immunogenic formulation) supplies one or more immunogens. A formulation may be simple (a recombinant immunogen in homogeneous form) or complex (a killed or attenuated pathogenic cell). The formulation may be cellular or acellular. If acellular, the immunogen may be unbound, or bound to a carrier. The immunogen may also be naturally produced, or synthetic, the latter including recombinant immunogens.

A physician may substitute a live vaccine with a killed vaccine or killed vaccine for a live vaccine. The physician may administer a vaccine without an adjuvant for example a tetanus toxoid instead of a tetanus vaccine with an aluminum adjuvant or a tetanus vaccine mixed with diphtheria toxoid. The physician may administer an acellular pertussis vaccine as compared to a whole cell pertussis vaccine. The physician may administer a diphtheria tetanus vaccine instead of a diphtheria, tetanus, pertussis vaccine.

The current invention could be modified for other high risk subjects. For example those at increase risk of metabolic syndrome based on genetic screening or family history. In this case the relative risk may be the same as the general population but the attributable or absolute risk will be higher. Alternatively, an individual with a history of either type II diabetes, insulin resistance, microalbuminurea, hypertension, hyperlipidemia and or hypertriglyceridemia, low HDL cholesterol, or obesity is at increased risk for developing a secondary problem or metabolic syndrome. While the relative risk may be the same as in the general population the attributable or absolute risk will be increased.

There are a number of alterations of these methods and combinations of methods that one skilled in the art may use. It is the intention of the inventor to include these.

### Kits

The present invention also encompasses kits for administration of one or more vaccine immunogens according to the methods of the present invention. A kit typically comprises one or more receptacles, each receptacle comprising an immunogenic composition (vaccine) which in turn comprises one or more immunogens, together with directions for use and warnings concerning possible adverse effects.

The improved kits contain specific information that would lead the immunizer to inquire about the family history or medical history relevant to the risk of metabolic syndrome and or one of its components a chronic immune mediated disorder prior to immunizing the recipient. The information may include warning about the risk of metabolic syndrome and or include suggestions on way to avoid the risk.

The kit may contain information that the risk of metabolic syndrome or one of its components is elevated in those receiving vaccines and there is a scheduling/dosing effect. Preferably, the kits would contain information on the risks of developing metabolic syndrome or one of its components with different immunization schedules. For example the kit may provide information on a dosing effect, e.g., that the more doses of the vaccine given after 8 weeks is associated with an increased risk. The kit may provide information on the risks of two different vaccines that protect against the same disease so that the health care provider or user can better choose which product to use. For example the kit may provide information on both a whole cell and non whole cell pertussis vaccine or on both a live and killed polio vaccine.

### Immunization Schedule

An immunization schedule is a program for the administration of one or more specified doses of one or more specified immunogens, by one or more specified routes of administration, at one or more specified ages of the immunization subject and usually includes a specific time interval between doses. A supplemental immunization schedule is one intended to supplement a standard immunization schedule which is commonly followed in the region in which the subject resides.

While the immunogens which can be administered according to the present invention are discussed in detail in a later section, certain prefatory remarks regarding their place in the immunization schedule are appropriate here. The immunization schedule may provide for one or more administrations of a single immunogen, multiple immunogens which collectively immunize against the same or different strains for the same infectious disease, or multiple immunogens which collectively immunize against a plurality of different infectious diseases. The immunogens may be separately or simultaneously administered, and, in the latter case, may be combined into a single pharmaceutical composition for ease of administration.

### Immunogens

It is the intent of the inventor to limit this invention to vaccine immunogens used to prevent infectious diseases, cancer immunogens to treat specific cancers, and or immune modulators used to stimulate the immune system or specific parts of the immune system such as the humoral (antibody), the cellular, the natural killer, or the phagocytic. The following definitions help differentiate vaccine immunogens which are designed to prevent against specific infections versus immune modulators which are used to treat infections such as infections with viral hepatitis or cancers.

Immunogens correspond to a class of molecules that elicit an immune response through classical immunologic pathways as in the non-limiting example of the incorporation in an MHC molecule of an antigen processing cell where the immunogens can potentially interact with antigen specific T cell receptors. Alternatively, as another non limiting example, immunogens can bind to antigen specific binding regions of immunoglobulins which may lead to stimulating the B lymphocytes (if on the surface of B lymphocytes), but alternatively could elicit an immune response through other means, e.g., by the activation of complement, or the modulation of Fc receptors.

An immunogen of the present invention is a molecule which may be derived from a virus, bacteria, yeast, mold, plant, insect, cancer cells, allogeneic or xenogeneic animal or compound or composition that immunologically cross reacts with a naturally occurring immunogen. Such agents may be made from the killed or live bacteria, killed or live viruses, recombinant or chemically synthesized or purified immunogenic agents including antigens, fragments or cross reacting synthetic or recombinantly produced peptides, carbohydrates, lipids or any combination thereof. Such agents can be combined with each other and with vaccines against infectious diseases to substantially prevent or reduce the incidence of immunologic disorders according to the present invention. The term “immunologically cross reacts” refers to molecules that induce antibodies or T-cells that bind to the cross reactive molecule or fragments thereof.

Weak immunogens may be limited to the ability to invoke changes in such immune mediator cells, such as lymphocytes (B and/or T cells), macrophages and natural killer cells, such as the release of lymphokines, altered cell movement, or altered composition of cell surface receptors. Strong immunogens have the additional ability to cause either an humoral immune response (such as, e.g., antibodies to said agent) or a cellular immune response (such as, e.g., a delayed type skin reaction to said agent).

There are several examples of conventional immunogens. The classical example is that of vaccines as in human vaccines. Such vaccines may be classified as living where such agents may multiply or perform homeostatic metabolic activity in the recipient, as in the live oral polio, live BCG, and live small pox vaccines, as non-limiting examples. Alternatively, conventional vaccines can be classified as inactivated (killed), where such agents have lost their ability to multiply or maintain homeostatic metabolic activity. Non-limiting examples of such killed vaccines include tetanus toxoid, diphtheria toxoid, and the killed whole cell pertussis vaccine. Other non-limiting examples of conventional non-living immunogens are haptens, anti-idiotype antibodies, and nucleic acid molecules, such as DNA or RNA, that can be expressed in cells as immunogenic molecules encoded by such nucleic acids. Alternatively, conventional immunogens may be classified according to their functional or structural properties in a microorganism such as capsular, fimbriae, nuclear, cell wall, membrane, and cytoplasmic immunogens. Chemically speaking, immunogens of biological origin are most often peptides (including proteins), carbohydrates, glycopeptides, lipids, glycolipids, or lipopeptides.

Immunogens of the present invention may be pediatric or non-pediatric immunogens. The term “pediatric immunogens” refers to immunogens that after birth were routinely administered in 2006 to children less than one year old, in modern developed nations of moderate latitudes. These agents include but are not limited to BCG, measles, mumps, rubella, diphtheria, pertussis, hemophilus influenza, tetanus, hepatitis A, hepatitis B, varicella, influenza, pneumococcus, meningococcus, rotavirus and polio. Other immunogens are considered non-pediatric immunogens, and may include, but are not limited to, the group consisting of anthrax, plague, encephalitis, Hepatitis C, hepatitis e, typhus, typhoid fever, streptococcus, staphylococcus, lyme disease, cholera, campylobacter, helicobacter, E. coli, shigella, leishmania, leprosy, cytomegalovirus (CMV), human papilloma virus, respiratory syncytial virus, Epstein Barr virus, herpes, parainfluenza, adenovirus, human immunodeficiency virus (HIV), hepatitis A, NonA NonB hepatitis, rabies, yellow fever, rabies, Japanese encephalitis, flavivirus, dengue, west nile virus, avian flu virus, SARS coronaviruses, toxoplasmosis, coccidiomycosis, schistosomiasis, and malaria immunogens.

The term immunogen used here is not intended to cover foods customarily given for nutritional reasons to infants and other children, such as bovine milk, common baby formula, and common baby food, even though such foods may nominally contain “immunogens”. It should be noted that researchers are developing edible vaccines comprising genetically engineered food protects that also protect against traditional infections such as polio. In such a case, the edible vaccine components would be considered immunogens.

Immunogens are distinct from immune modulators. There are several classes of immune modulators. One class is “immunocyte receptor ligands.” Members of this class of agents bind to cell receptors of immune mediator cells in a non-antigen specific manner to cause the induction of an immune response, e.g., as defined herein. One subclass of this group is cytokines. Cytokines that are produced by lymphocytes are termed lymphokines, whereas peptides produced by monocytes or macrophages are given the term monokines. Thus, the terms cytokines, lymphokines, and interleukins may be used interchangeably to designate those peptide molecules that modulate host responses to foreign antigens or host injury by regulating the growth, mobility and differentiation of leukocytes and other cells.

Known cytokines include interleukins (IL) IL-1 (also endogenous pyrogen (EP), lymphocyte activating factor (LAF), mononuclear cell factor, catabolin, osteoclast activating factor and hematopoetin 1) IL-2 (also T cell growth factor, (TCGF)), IL-3 (multicolony stimulating factor (M-CSF), P-cell stimulating factor, WEHI-3B factor, mast-cell growth factor and histamine-producing factor), IL-4 (B-cell growth factor (BCGF), B-cell stimulatory factor-1 (BSF-1), IL-5 (T-cell replacing factor (TRF), B-cell growth factor II (BCGF-II), eosinophil differentiation factor (EDF), IL-6 (.beta.sub.2 inteferon (IFN-.beta.sub.2), B-cell stimulating factor 2 (BSF-2), 26-kDa protein, hybridoma/plasmacytoma growth factor (HPGF or IL-HP-2), hepatocyte stimulating factor (HSF), and T-cell activating factor (TAF)), IL-7, IL-8 (neutrophil activating protein 1 (NAP-1), IL-10 (also cytokine synthesis inhibitory factor (CSIF); tissue necrosis factors (TNF) TNF.alpha. (also lymphotoxin (LT) and TNF.beta. (also macrophage derived TNF); interferons (IFN) IFN.alpha. and IFN.beta. (also type I IFN) and IFN.gamma. (also type H IFN) and tissue growth factor (TGF).beta.

Granulocyte-Macrophage Colony Stimulating Factor or GM-CSF is another non-limiting example of a cytokine in this class that causes the production of macrophages. Thymic hormones both natural and synthetic derivatives are another non-limiting example of receptor ligand class of immune modulators, they are an example of a subclass of receptor ligands that cause division of immature immune mediator cells, in this case thymocytes or premature lymphocytes.

Cytokines modulate target cells by interacting with cytokine receptors on the target cell. Principal cell sources of cytokines include T lymphocytes, B lymphocytes, macrophages, stromal cells, monocytes, leukocytes, and platelets. While cytokine specific receptors are specific for a given cytokine, cytokine receptors are grouped into families based on shared features. The first group of cytokine the hemopoetin group which include immune system cells that bind IL-2, IL-3, IL-4, IL-6 and IL-7. A second receptor family is the TNF receptor family which bind both TNF.alpha. and TNF.beta. A third family is the immunoglobulin (Ig) superfamily receptor family, which contains an Ig sequence like motif and includes human IL-1 and IL-6 receptors.

See, e.g., Dawson, In Lymphokines and Interleukins (Dawson, ed.) CRC Press, Boca Raton, Fla. (1991); Mosmann et al, Immunol. Rev. 123: 209-229 (1991); Mosmann et al, Immunol. Today 12:A59-A69 (1991); Sherry et al, Curr. Opinion Immunol. 3:56-60 (1991); Paul, Blood 77:1859-1870 (1991); Dower et al, J. Clin. Immunol. 10:289-299 (1990).

A second subclass in this receptor ligand group include lectins. A non-limiting example of a receptor ligand is a monoclonal antibody or fragment capable of binding and/or modulating a receptor, e.g., as a T cell receptor or an IL-2 receptor.

A second class of immune modulators are anti-receptor molecules. These agents can cause the production of antibodies or T cells that can either block receptors on a cell surface or kill such cells in a recipient. As non-limiting examples, one skilled in the art could induce active immunization in a recipient leading to the formation of antibodies or cytotoxic T cells that could, e.g., block lymphokine receptors on a cell, neutralize certain subtypes of antibodies, kill B lymphocytes that make certain subtypes of antibodies, or kill T lymphocytes that have a certain subtype of receptors on their surface.

A third class of immune modulators are transplanted cells, which may include immune mediator cells as defined above that can induce responses by releasing lymphokines or by secreting other molecules. As non-limiting examples these cells can be lymphocytes, macrophages, splenocytes and/or thymocytes. As non-limiting examples the products the transplanted cells release can be lymphokines, as well as heterogenic or allogenic molecules as in proteins, carbohydrates and lipids.

A fourth class of immune modulators are “general immune modulators.” These agents also go by other names including immune modulating agents or immune response modulators. Depending on what dose and how these agents are given they can be termed immune potentiators or immunosuppressants. These agents often have the ability to cause non-antigen specific activation of immune mediator cells through the release of lymphokines compared to vaccines that usually cause specific activation of clones with specific affinity for the vaccine antigens. General immune modulators are often used at higher doses than conventional immunogens and/or are typically given at frequent intervals as in every seven days or less. These agent often provide no protection against common pathogens in contrast to many vaccines and other pharmaceutically acceptable conventional immunogens. These agents generally do not employ adjuvants in contrast to vaccine immunogens. Non-limiting examples of immune modulators include the biologic OK-432 and the chemical entities levamisole and isoprinosine.

It should be noted that some agents may be members of more than one of the above groups or classes. As a non-limiting example, a lymphokine may be a conventional immunogen if it is derived from a heterologous species to which it is given as in the case of administering a mouse derived lymphokine to a human. Hybrid or fusion molecules can also be made that contain an biologically active part that has lymphokine activity and another section that contains an conventional immunogen.

### Immunogenic Agents

An immunogenic agent (vaccine, immunogenic formulation) of the present invention is a pharmaceutically-acceptable composition comprising at least one immunogen in an amount such that, when administered according to an immunization schedule as disclosed above, it contributes to the desired effect against a chronic immune-mediated disorder, and also against an infectious disease. When multiple immunogens, and/or multiple dosings of the same immunogen, are administered, the individual doses of individual immunogens may by themselves be subimmunogenic, provided that in aggregate, when administered according to the schedule, an immunogenic effect is achieved.

A hepatitis B immunogen may be recombinant or produced from blood products. A polio immunogen may be a live and or killed immunogen. A polio vaccine may be trivalent such that the vaccine may induce in mammals antibodies reactive to three serotypes of polio virus. A hemophilus influenza, meningococcus and pneumococcus immunogen may be a conjugated or unconjugated immunogen. A pertussis immunogen may be a non-whole cell or a whole cell immunogen. The non-whole cell immunogen may include a cellular pertussis immunogen.

In addition to the immunogen, the pharmaceutical composition may contain suitable pharmaceutically acceptable carriers, such as excipients, carriers and/or auxiliaries which facilitate processing of the active compounds into preparations which can be used pharmaceutically. Such carriers may include depot adjuvants that release an immunogen in vivo over a prolonged period as compared to administration of an unbound immunogen. Preferably the depot adjuvant comprises an aluminum, calcium or salts thereof, such as aluminum sulfate (alum), aluminum phosphate, calcium phosphate or aluminum hydroxide.

Pharmaceutical compositions comprising at least one immunogen useful according to the present invention may also include suitable solutions for administration, intramuscularly, intravenously, subcutaneously, dermally, orally, mucosally, or rectally or by any other injection, and contain from about 0.001 to 99.999 percent, preferably from about 20 to 75 percent of active component (i.e. the immunogen) together with the excipient. Compositions which can be administered rectally include suppositories. Preparations of immunogenic agents for parenteral administration include sterile aqueous or non-aqueous solutions, suspensions, and emulsions, which may contain auxiliary agents or excipients, such as suitable adjuvants, which are known in the art. Pharmaceutical compositions such as tablets and capsules can also be prepared according to routine methods. See, e.g., Berker, infra, Goodman, infra, and Avery, infra, which are entirely incorporated herein by reference, including all references cited therein.

The immunogenic agents of the present invention may optionally include immunomodulators other than immunogens. Such immunomodulators may also be administered separately as a part of the program. The compositions of the present invention may also include pharmaceuticals whose primary activity is non-immunological.

It is understood that the dosage of an immunogenic agent of the present invention administered in vivo or in vitro will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. In the context of the present invention “one dose” may include concurrent or separate administration of more than one immunogen comprised of an immunogenic agent according to the present invention. See, e.g., Berkow et al, eds., The Merck Manual, 16th edition, Merck and Co., Rahway, N.J., 1992; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. 1987), which references and references cited therein, are entirely incorporated herein by reference.

The total dose, as in a pharmaceutically acceptable dose, required for each treatment may be administered by multiple doses or in a single dose. An immunogenic agent may be administered alone or in conjunction with other therapeutics directed to immunologic disorders, such as allergies, immune mediated cancers and autoimmune pathologies, as known in the art.

The pharmaceutically acceptable dosage of the immunogen will usually be about 0.01 .mu.g to about 5 mg of immunogen, per kg body weight, and preferably from about 0.1 .mu.g/kg to about 1 mg/kg body weight, still more preferably about 1 .mu.g/kg to about 300 .mu.g/kg, most preferably about 10 .mu.g/kg to about 100 .mu.g/kg. Nevertheless, Applicants' invention is not limited to the dosages set forth above. The active agent is the at least one immunogenic agent that induces an immune response according to the present invention. The safe dose will vary depending on the agent. Some immunogens are toxic at low doses while others are not.

### Pharmaceutical Purpose

The immunogenic agents of the present invention, whether or not incorporated into kits, may be pharmaceutically administered, according to an immunization schedule, to achieve a pharmaceutical purpose. One pharmaceutical purpose of the invention is to protect subjects against at least one infectious disease, by administering one or more immunogens which elicit a protective immune response against such disease. The term “protection”, as used herein, encompasses “prevention,” “suppression” or “treatment.” “Prevention” involves administration of a pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.”

The “protection” provided need not be absolute, i.e., the disease need not be totally prevented or eradicated, provided that there is an improvement, preferably of a statistically significant character (p=0.05,) relative to a control population. Protection may be limited to mitigating the severity, rapidity of onset or duration of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage. The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk. Prophylaxis may be rendered to those considered to be at higher risk for the disease by virtue of family history, prior personal medical history, or elevated exposure to the causative agent.

The second pharmaceutical purpose of the invention is in Prophylaxis against at least one component of metabolic syndrome. To the extent that administration of the vaccine is a risk factor for developing metabolic syndrome or its components, the first and second purposes are at odds. In this situation, the invention attempts to balance the risks of developing the infectious disease and developing the metabolic syndrome or its components. That is, the invention attempts to achieve a clinically acceptable level of protection against the infectious disease while at the same time achieve a reduced risk of developing metabolic syndrome or its components, at least relative to the risk with an alternative immunization schedule.

### Pharmaceutical Administration

The immunogenic agents of the present invention may be administered by any effective route, for example, by various parenteral routes such as subcutaneous, intravenous, intradermal, intramuscular, intraperitoneal, intranasal, transdermal, or buccal routes. Alternatively, or concurrently, administration may be by the oral route. A preferred mode of using an immunogenic agent or composition of the present invention is by intramuscular application.

### Pharmaceutical Testing of Vaccines.

The enclosed invention includes improved methods of testing vaccines, immune modulators, and immunization schedules for their ability to increase the risk of metabolic syndrome and its components. Ideally one would compare different schedules which differ by the number of doses of one or more immunogens, composition of vaccines including a live versus a killed vaccine (for example polio) or an acellular versus whole cell (for example pertussis) or an conjugated versus unconjugated vaccine (for example hemophilus or pneumococcus or meningococcus) or a combined versus single vaccine (for example separate measles, mumps, rubella vaccines as opposed to a combined vaccine), the presence of one or more immunogens (for example a 3 valent meningococcal vaccine versus a 2 valent meningococcal vaccine), the amount of immunogen administered, the timing of the schedule (for example starting at birth or later). A proper test should ideally follow the recipient to ensure the effect is not transient but prolonged. Ideally this time after immunization is at least an interval of 1 month, more preferably 2 months, more preferably 3 months, more preferably 4 months, more preferably 6 months, more preferably 8 months, more preferably 1 year, more preferably 2 years, more preferably 3 years, more preferably 4 years, and more preferably 5 years or more. Preferably the study should include at least a group size of 20 patients, more preferably 50 patients, more preferably 100, more preferably 200, more preferably 400, more preferably 600, more preferably 800, more preferably 1 thousand, more preferably 2 thousand, more preferably 4 thousand, more preferably 8 thousand, more preferably 10,000, more preferably 20,000, more preferably 30,000, and even more preferably 40,000 or more. Preferably several endpoints should be studied comprising one or more of the following metabolic syndrome, obesity, hypertension, hypertriglyceridemia, low HDL, microalbuminurea, and insulin resistance. Preferably the results are statistically significant however statistical significance is not necessary to indicate risk. Preferably the data should be analyzed for confounding variables and previous medical or family history.

Ideally one can study the effect of vaccines in race specific groups or groups with family histories of specific disorders (for example type 1 diabetes, type 2 diabetes, metabolic syndrome). For example it is now shown that Japanese tend to develop type 2 diabetes (and metabolic syndrome) more commonly following immunization while Whites tend to develop type 1 diabetes following immunization and less commonly type 2 diabetes or metabolic syndrome. Power is important in detecting an statistically significant effect, and the power is greater in populations where the incidence of the adverse event is higher. In this case the incidence of metabolic syndrome, type 2 diabetes and related disorders is greater in Asians, American Indians, Blacks, Polynesians (including Maoris), and Australian Aboriginals compared to Whites. Analysis of the effect of vaccines on metabolic syndrome, type 2 diabetes, and related conditions can be best performed by an analysis in these high risk groups. Furthermore some families are at increased risk of type 1 diabetes because they secrete lower amounts of cortisol while other families have low risks for type 1 diabetes but high risk for metabolic syndrome, type 2 diabetes and related conditions because they secrete increased amounts of cortisol. It has now been shown (below) that Japanese are at high risk for developing type 2 diabetes following BCG immunization which has been linked to their increased cortisol release following immunization. By studying the effect of vaccines on metabolic disease, type 2 diabetes and related conditions in those with a family history of the disorder or a family history of increased cortisol release (following immunization for example) it will be easier to show an statistically significant effect.

Additional information on testing vaccines for risk has been previously published and is incorporated by reference (U.S. Pat. Nos. 5,723,283; 5,728,385; 6,420,139; 6,584,472; 6,638,739; 7,008,790; PCT/US 2005/011386 or WO 2005/097188 A3).

Having now generally described the invention, the same will be more readily understood through reference to the following examples which are provided by way of illustration, and not intended to be limiting of the present invention.

### Supporting Data

Associations between vaccines and different diseases could indicate causation or they could happen simply by chance. Risk exists when there is an association unless causation is ruled out. A set of criteria have been used to indicate the likelihood that associations such as the association between immunization and metabolic syndrome described here, are the result of a causal relationship. These criteria were published by Sir Austin Bradford Hill (32). The evidence supporting an causal relationship between immunization and metabolic syndrome are included. The evidence include a plausible mechanism of action and other indirect evidence that is part of the criteria set up by Sir Austin Bradford Hill. Epidemiology data is included. Finally, and for the first time, the evidence supporting a causal relationship between immunization and metabolic syndrome or its components are analyzed according to the Austin Bradford Hill criteria.

### I. Metabolic Syndrome Epidemiology

Metabolic syndrome is a common disorder which is associated with an increased mortality. Its prevalence is rising rapidly making it a major heath concern. The syndrome comprises hypertension, hyperlipidemia and or hypertriglyceridemia, low HDL cholesterol, insulin resistance, microalbuminurea and obesity. The criteria needed to be defined as having the syndrome has been defined differently by different groups which alters the prevalence of the disease (7). The prevalence in the US has been estimated as 27% in adults (5) and 6.4% of adolescents (6). There is significant racial difference.

Epidemic: There is an epidemic on metabolic syndrome and the individual components that collectively define metabolic syndrome. In US adolescents the prevalence has risen from 4.2% (1988-1992) to 6.4% (1999-2000) (6) (p<0.001). In adults (5) metabolic syndrome has risen much more slowly from 24% (1988-1992) to 27% (1999-2000) 0.088) with most of the rise seen in women. The rise is primary attributable to increases in blood pressure waist circumference and hypertriglyceridemia. Metabolic syndrome outside the US has also risen including Canada (33).

Obesity, one of the components of metabolic syndrome, is epidemic in adolescents (34, 35). It has risen from 5.5% to 15.5% of US adolescents from 1976-2000. Obesity has risen in children drastically around the world including Finland (33). Obesity at age 7 is closely associated with development of metabolic syndrome later in life (36). Blood pressure has also risen in adolescents (37). Hypertension and obesity are associate and some have indicated they believe that obesity causes hypertension (37, 38), however there is data to indicate the epidemic in hypertension is not caused by the obesity (39).

Risk of mortality: One of the main concerns about the epidemic of metabolic syndrome is the risk for developing other diseases and mortality. Metabolic syndrome is associated with an increased risk of developing type H diabetes (8), coronary vascular heart disease (9) and death (9).

Diet and exercise: Poor diet and lack of exercise have been blamed for the epidemic of metabolic syndrome. It has been proposed to improve diet and exercise as a treatment and prevention. However this has not been very effective (11). Exercise can increased calorie utilization and can cause weight loss if calorie intake is constant. A low calorie diet can also lead to weight loss and help control lipid levels however failure rates are high in people suffering from obesity alone and have not been successful in stemming the epidemic of metabolic syndrome as the figures show. One problem with the approach is that it may not address the true pathophysiology of the disease. While diet and exercise will affect metabolic control they will generally be ineffective treatments if there is an underlying metabolic force driving a catabolic state. The best documented such state is the weight gain from prednisone and other corticosteroids. As discussed below there is now evidence that an vaccine induced inflammation is inducing excessive cortisol activity, leading to an epidemic of metabolic syndrome.

### IIA. Metabolic Syndrome an Inflammatory Condition

It has been proposed that metabolic syndrome is an inflammatory condition (40). This belief is supported by studies which have shown that inflammation predates metabolic syndrome (41, 42). A study on Finnish middle age men (41) found men with elevated CRP concentrations have higher age-adjusted risk of developing the metabolic syndrome. A study of men and women in Mexico (42) found women with elevated CRP in the highest tertile had an increased relative risk of developing the metabolic syndrome by 4.0.

Some have suggested that metabolic syndrome causes inflammation (43) which may occur, however inflammation has been independently and prospective associated with the development of components of metabolic syndrome independent of the presence of characteristics of metabolic syndrome (41, 42). Glucose intolerance/type 2 diabetes (44) and hypertension (45, 46) are both independently associated with inflammation. There is additional evidence that the once metabolic syndrome begins it causes more inflammation (43) which intern makes the disease worse. Adiposites and the accompanying macrophages appear to make inflammatory mediators. However while there is an association between obesity and inflammation (47), obesity can exist without inflammation in individuals with a healthy metabolic profile (48). The later evidence supports the view that the inflammation causes the obesity and not the obesity causing the inflammation.

### IIB. Metabolic Syndrome and its Similarity to Cushing's Disease

Metabolic syndrome has a remarkable similarity to patients with mild Cushingoid Syndrome (49, 50). There is direct evidence showing increased cortisol level associated with metabolic syndrome (51). Some have suggested that metabolic syndrome is impart due to increased cellular up take of cortisol (52). Exogenous glucocorticoid steroids are known to cause hypertension, obesity, hyperlipidemia, and in glucose intolerance. The effect is dose dependent. There are many similarities between excessive cortisol and metabolic syndrome. Cortisol is associates with cardiovascular events (53, 54) just like metabolic syndrome. Cortisol is associated with metabolic disturbances including increased glucose levels and hyperlipidemia (55) just like in metabolic syndrome.

### III. Inflammation and Cortisol Release

There is biological evidence that specific lymphokines released during inflammation can cause the release of cortisol and cause the biological changes that occur in metabolic syndrome. It has even been hypothesized (56) that the responses seen to lymphokines are acutely advantageous to help the host survive noxious events. Hyperlipidemia for example may help the body clear a fat soluble toxin. The problem appears that when inflammation becomes chronic in some individuals, the changes lead to metabolic syndrome. In this case changes that are a survival advantage acutely are chronically an hazard.

There is evidence that cytokine production, particularly IL-6, increases with age (28, 57, 58) and this can explain the increase in metabolic syndrome with age. Both IL-1 (59, 60) and IL-6 (61-63) enhances cortisol release and thus have the potential to cause a disease similar to metabolic syndrome. In addition IL-6 has been directly associated with the development of diabetes (44), insulin resistance (64) and altered lipid levels (65-67).

### IV. Vaccines as a Contributing Cause of Inflammation, Cortisol Release and Metabolic Syndrome.

It has been previous shown that vaccines are the major contributor of a rise in type 1 diabetes in Western countries (2, 3). No one has ever linked immunization to metabolic syndrome or to the chronic components of metabolic syndrome such as obesity, hypertension, hyperlipidemia and or hypertriglyceridemia, low HDL cholesterol, and insulin resistance. Evidence below shows that vaccination can also explain the epidemics of type H diabetes (68) and metabolic syndrome (6) in children which mimic the epidemics of Type 1 diabetes mellitus (69) known to be caused by vaccines.

The acellular diphtheria tetanus pertussis vaccine has been reported to causes the release of IL-6 (70). The influenza vaccine stimulates release of IL-6 and IL-10 (71). The DT-Polio-Typhim vaccine stimulated IL-6 production (72). The DTwP elicited 11-6 but not the DTaP at 2 days (73). The tetanus vaccine has also been shown to stimulate the release of cortisol (74). Several papers have shown that immunization of children can alter cortisol release at least in the short term (19-24).

Immunization in the first year of life may affect the onset of metabolic syndrome by resetting of the hypothalamus, creating more cortisol release. Data shows that children who undergo stress in utero for example as demonstrated by low birth weight, produce higher cortisol release and hence have increased symptoms resembling metabolic syndrome (75-77). Data shows that children which are low birth weight are more likely to develop metabolic syndrome (78).

Additional Applications of these Findings.

The findings below show that immunization is associated not only with autoimmunity but also with metabolic syndrome. Administration of cortisol inhibitors to an individual will decrease symptoms and component diseases/disorders of metabolic syndrome but increase the risk of autoimmunity. Warnings should be given to those taking steroid inhibitors to look for signs of autoimmunity as well as check blood tests for autoimmunity including titers of autoantibodies. Treating chronic immune mediated disorders induced by immunization with steroids such as prednisone and other glucocorticoids can lead to the development of metabolic syndrome and its component diseases/disorders. One should warn about the risk of treating vaccine adverse events with glucocorticoids and related agents (ACTH for example) leading to type 2 diabetes, metabolic syndrome, and its component diseases/disorders including hypertension, obesity, insulin resistance, altered HDL, dyslipidemia, microalbuminurea and kidney disease for example. Those with increased inflammation post immunization, including those with chronic inflammation post immunization lasting more than 2 months or more than 4 months or more than 8 months or more than 1 year, or more than 2 years or more than 5 years can take non glucocorticoid anti-inflammatory agents to decrease the risk of type 2 diabetes, metabolic syndrome, and component diseases/disorders.

Warnings regarding immunization can be directed to specific high risk groups. For example racial groups (some call these ethnics groups and the terms may be used interchangeably) Asians, American Indians, Hispanics, Blacks, Australian Aboriginals, and Polynesians (including Maoris) can be warned that they are at increased risk of vaccine induced type 2 diabetes, metabolic syndrome, and its component diseases/disorders (hypertension, obesity, insulin resistance, altered HDL, dyslipidemia, microalbuminurea and kidney disease for example) following immunization than Whites. Whites can be told they are at increased risk for type 1 diabetes and autoimmune disorders following immunization compared to other racial groups. Likewise those with increased cortisol secretion (including those with increased secretion after immunization compared to vaccinated controls) can be told they are at increased risk of vaccine induced metabolic syndrome, type 2 diabetes and related disorders. Individuals can be tested for cortisol activity (including those with increased or decreased secretion after immunization compared to vaccinated controls) to determine those who are increased risk for metabolic syndrome (for example high cortisol activity including those who secrete more cortisol and those who respond more strongly to a given dose of cortisol) and those at increased risk for autoimmunity (low cortisol activity). Cortisol or any similar marker can be used including ACTH, serum, plasma, urine, salivary cortisol or metabolite or precursor of cortisol or related molecule. One skilled in the art will know what tests will give comparable or useful information similar to what would be provided by a cortisol test. Markers of cortisol release can be used instead of cortisol including C-reactive protein, IL-6, and other lymphokines mentioned above that correlate with vaccine induced inflammation. Those with increased markers of inflammation following immunization are at increased risk for vaccine induced type 2 diabetes, metabolic syndrome, and related disorders as well as autoimmune and chronic immune mediated disorders.

The findings of this research indicate vaccines increase the risk of multiple components of factors that cause atherosclerotic disease. Type 2 diabetes, metabolic syndrome, and its component diseases/disorders (hypertension, obesity, insulin resistance, altered HDL, dyslipidemia, microalbuminurea and kidney disease all increase the risk of atherosclerotic disease. Subjects contemplating receiving a vaccine should be warned that the vaccination may increase the risk of atherosclerotic disease leading to heart attacks, strokes, kidney failure, amputations and aneurysms for example. Vaccines will increase the need for bypass surgery and angioplasty. Subjects receiving vaccines will need more diagnostic procedures such as angiography (both invasive and non invasive such as spiral CT). Vaccines should be tested for their effect on atherosclerotic disease by performing clinical trials, epidemiological studies or animal toxicity studies on new onset and worsening of existing atherosclerotic disease. The end point of such studies could include assessment of evidence of atherosclerosis in arteries (for example use of angiography) or the clinical complications of atherosclerosis (for example heart attacks, strokes, aneurysms, claudication, amputations, gangrene, bypass surgery, angioplasty, kidney failure/transplant, slow or non healing ulcers, skin grafts).

### Epidemiology Data

The data below shows that there is an association between the number of vaccine doses recommended and rise in obesity and metabolic syndrome in both the US and Finland.

### US:

The number of vaccines, vaccine doses, and vaccine immunogens (valence) recommended in the US is increasing on a regular basis. This rise is associate with the rise of metabolic syndrome and obesity in the US (table 1). The results are statistically significant. Metabolic syndrome increased on average of 4% a year in adolescents 12-19 in US NHANES data from 1988-1992 to 1999-2000 (6) (p<0.001). Obesity in US children aged 4 to 12 increased on average of 3.23-5.85% per year depending on race from 1986-1998 in the National Longitudinal Survey of Youths (79). Overweight increased 1.41% to 3.60% per year depending on race in the same group. Similar rises were seen in children age 0-19 in the NHANES study (35, 80). Between 1988 and 2000 the prevalence of obesity rose 4.4% per year in children age 12-19, 3.4% year in children age 6-11, 4.2% per year in children age 2-5. Blood pressure also rose in children and adolescents between 1988 to 2000 (37) (p<0.001). In comparison Type 1 diabetes increasing 3% per year globally (81). There are no comprehensive diabetes registries in the US.

### Finland

Data from Finnish children (33) shows prevalence of obesity in children age 12-18, 1977-1999 increased 1.1% to 2.7% in boys (RR 2.45) and 0.4-1.4% in girls (RR 3.5); on average 5% per year. The prevalence of overweight increased from 7.2% to 16.7% in boys and 4.0% to 9.8% in girls, on average a 4% rise per year. This figure is very similar to the 3.4% per year increase in type 1 diabetes, 1965-1996 (69) which has been attributed to vaccines (2, 3). The number of vaccines given in Finland has also increased with obesity just like in the US (2, 3)

### Japan

BCG immunization was routinely given to Japanese elementary school children age 6 to 7 and junior high school children age 12 to 13 since prior to 1982. Immunization was discontinued at the end of 2002 (82). BCG immunization rates were provided by The Research Institute of Tuberculosis, JATA. The incidence of type 2 diabetes in all elementary and all junior high school students in Tokyo Japan has been recorded since 1974 (83-85) and verified data has been published through 2004 (85). Data on the incidence of type 2 diabetes was analyzed to determine if the incidence declined after discontinuation of BCG vaccine. Only incidence data of type 2 diabetes after 1982 was used because the age of BCG immunization changed in 1982 and this may explain the decreased incidence of diabetes prior to this time. Statistics were performed using Epiinfo 2000, CDC, Atlanta Ga. A two stratification analysis was performed with one stratification using data from elementary school children and one stratification using data from junior high school students. Mantel-Haensel Weighted Relative Risk calculation was performed and Greenland/Robins Confidence Limits created.

The results (Table 2) show that BCG immunization was associated with an relative risk of 2.78 (1.03<RR<7.48). Japanese have increased cortisol secretion following immunization compared to Whites (86) and this explains why Japanese have higher rates of type 2 diabetes but lower rates of type 1 diabetes than Whites (81, 84, 87, 88).

### Racial Differences

There is an global epidemic of both type 1 (81) diabetes, an autoimmune disease, and type 2 (89) diabetes in children. Epidemiology data comparing the incidence of both type 1 and type 2 diabetes in different racial populations of children were sought to help discern the cause of the simultaneous epidemics of type 1 and type 2 diabetes. Medline was searched to find papers on the incidence of both typed and type 2 diabetes in children. Key search words included type 1 diabetes, type 2 diabetes, incidence, children, adolescents. Abstracts and papers were read to find papers that contained incidence of both type 1 and type 2 diabetes. Incidence data was matched by age to allow more accurate comparisons. Statistics were performed using software Satistica, Stat Soft, Tulsa, Okla. Pearson Product-Moment Correlation was used to asses a possible correlation between the risk of type 1 diabetes and a risk of type 2 diabetes.

Three papers were found containing studies measuring the incidence of both type 1 and type 2 diabetes in children where the results were recorded by race. The studies included New Zealand (90) where the incidence was compared between Whites and Maoris (Polynesians), Australia (91) where the incidence was compared between Whites and Aboriginals, and the US (88) where the incidence was compared between Whites, African Americans, Asians and Polynesians, Hispanics, and American Indians.

Table 3 compares the incidence of type 2 diabetes and type 1 diabetes. The incidence of type 2 diabetes showed a negative correlation with the incidence of type I diabetes. The correlation coefficient was ±0.85 (p<0.05) in children aged 10-19 and ±0.73 in children age 0-14 (p<0.05).

### Austin Bradford-Hill

Austin Bradford-Hill's criteria are used to show whether an association is likely to be an causative relationship. A good review of when one can make the leap from association to causation is a 1965 article by Sir Austin Bradford Hill “The Environment and Disease: Association or Causation” (32). He mentions nine factors, eight of which are demonstrated above. This is strong evidence that the association is a causative relationship.

### Austin Bradford-Hill Criteria

### 1. Strength.

Answer: Yes. The association is strong and in children under 6 months of age the rise of obesity can not be explained by competing theories of poor diets and lack of exercise. The drop in type 2 diabetes in Japan following the discontinuation of BCG immunization is unprecedented. Only immunization has been published to cause such an effect on diabetes.

### 2. Consistency.

Answer: Yes. The effect is consistent in different areas of the world including the US and Finland.

### 3. Specificity.

Answer: Yes. Exposure to vaccines has been linked to immunological disorders and not to unrelated disorders.

### 4. Temporality.

Answer: Yes. Children are immunized at 2 months of age and metabolic syndrome occurs later. The incidence of type 2 diabetes decreased after discontinuation of BCG vaccine in Japan.
5. Biological gradient.
Answer: Yes. There is a statistically significant correlation linking the number of vaccine doses to the prevalence of obesity.

### 6. Plausibility.

Answer: Yes. The proposed mechanism has been described.

### 7. Coherence.

Answer: Yes. There is an epidemic of obesity, hypertension, metabolic syndrome, type 2 diabetes and type 1 diabetes which increased with the increased number of vaccine doses recommended.
8. Experimental evidence.
Answer: Yes. Studies show increased inflammation and cortisol levels after immunization. An anti-inflammatory reduced the incidence of new cases of diabetes.

### 9. Analogy.

Answer: Yes. The findings are analogous to vaccine induced type 1 diabetes.

### REFERENCES

1. Classen D C, Classen J B. The timing of pediatric immunization and the risk of insulin-dependent diabetes mellitus. Infectious Diseases in Clinical Practice 1997:6:449-54.
2. Classen J B, Classen D C. Clustering of cases of IDDM occurring 2-4 years after vaccination is consistent with clustering after infections and progression to IDDM in autoantibody positive individuals. JPEM 2003:16:495-508.
3. Classen J B, Classen D C. Clustering of cases of insulin dependent diabetes (TDDM) occurring three years after Hemophilus influenza B (HiB) immunization support causal relationship between immunization and IDDM. Autoimmunity 2002:35:247-53.
4. Classen J B, Classen D C. Vaccines and the risk of insulin dependent diabetes (IDDM), potential mechanism of action. Medical Hypotheses 2001:57:532-38.
5. Ford E S, Giles W H, Mokdad A H. Increasing Prevalence of the Metabolic Syndrome Among US Adults. Diabetes Care 2004:27:2444-49.
6. Duncan G E, Li S M, Thou X H. Prevalence and Trends of a Metabolic Syndrome Phenotype Among US Adolescents, 1999-2000. Diabetes Care 2004:27:2438-43.
7. Ford E S, Giles W H. A Comparison of the Prevalence of the Metabolic Syndrome Using Two Proposed Definitions. Diabetes Care 2003:26:575-81.
8. Lorenzo C, Okoloise M, Williams K, Stem M P, Haffner S M. The Metabolic Syndrome as a Predictor of Type 2 Diabetes. Diabetes Care 2003:26:3153-59.
9. Katzmarzyk P T, Church T S, Janssen I, Ross R, Blair S N. Metabolic Syndrome. Obesity, and Mortality. Diabetes Care 2005:28:391-97.
10. Schulze M B, Hu F B. Dietary Approaches to Prevent the Metabolic Syndrome. Diabetes Care 2004:27:613-14.
11. McAuley K A, Williams S M, Mann J I, Goulding A, Chisolm A, Wilson N, et al. Intensive Lifestyle Changes are Necessary to Improve Insulin Sensitivity. Diabetes Care 2002:25:445-52.
12. Kim J, Petersen K E, Scanlon K S, Fitzmaurice G M, Must A, Oken E, et al. Trends in overweight from 1980 through 2001 among preschool-aged children enrolled in a health maintenance organization. Obesity 2006:14:1107-12.
13. Tsai M Y, Hanson N Q, Straka R J, Hoke T R, Ordovas J M, Peacock J M, et al. Effect of influenza vaccine on markers of inflammation and lipid profile. J Lab Clin Med 2005:145:323-27.
14. Doherty J F, Golden M H, Raynes J G, Griffin G E, McAdam K P. Acute-phase protein response is impaired in severely malnourished children. Clin Sci 1993:84:169-75.
15. van der Beek M T, Visser L G, de Maat M P M. Yellow fever vaccination as a model to study the response to stimulation of the inflammatory system. Vascular Pharmacology 2002:39:117-21.
16. Posthouwer D, Voorbij H A M, Grobbee D E, Numans M E, van der Born J G. Influenza and pneumococcal vaccination as a model to assess C-reactive protein response to mild inflammation. Vaccine 2004:23:362-65.
17. Hacker U T, Jelineck T, Erhardt S, Eigler A, Hartmann G, Nothdurft H D, et al. In vivo synthesis of tumor necrosis factor-alpha in healthy humans after live yellow fever vaccine. Journal of Infectious Diseases 1998:177:774-78.
18. Reinhardt B, Jaspert R, Niedrig M, Kostner C, L'age-Stehr J. Development of viremia and humoral and cellular parameters of immune activation after vaccination with yellow fever virus strain 17D: a model of human flavivirus infection. Journal Medical Virology 1998:56:159-67.
19. Gunnar M R, Brodersen L, Krueger K, Rigatuso J. Dampening of adrenocortical responses during infancy: normative changes and individual differences. Child Development 1996:67:877-89.
20. Lewis M, Ramsay D S. Stress reactivity and self-recognition. Child Development 1997:68:621-29.
21. Ramsay D, Lewis M. Reactivity and regulation in cortisol and behavioral responses to stress. Child Development 2003:74:456-64.
22. Lewis M, Thomas D. Cortisol release in infants in response to inoculation. Child Development 1990:61:50-59.
23. Lewis M, Ramsay D S. Developmental changes in infants' responses to stress. Child Development 1995:66:657-70.
24. Ramsay D S, Lewis M. Developmental change in infant cortisol and behavioral response to inoculation. Child Development 1994:65:1491-502.
25. Gherardi R K, Coquet M, Cherin P, Belec L, Moretto P, Dreyfus P A, et al. Macrophage myofascititis lesions asses long-term persistence of vaccine-derived aluminum hydroxide in muscle. Brain 2001:124:1821-31.
26. Authier F J, Cherin P, Creange A, Bonnotte B, Ferrer X, Abdelmoumni A, et al. Central nervous system disease in patients with macrophage myofascititis. Brain 2001:124:974-83.
27. Catania A, Airaghi L, Manfredi G, Zanussi C. Hormonal responses during antigenic challenge in normal subjects. hit J Neurosci 1990:51:296-.
28. Fagiolo U, Cossarizza A, Scala E, Fanales-Belasio E, Ortolani C, Cozzi E, et al. Increased cytokine production in mononuclear cells in healthy elderly people. European Journal of Immunology 1993:23:2375-78.
29. Wang C S, Wang S T, Lai C T, Lin L I, Lee C T, Chou P. Reducing major cause specific hospitalization rates and shortening hospital stays after influenza vaccination. Clinical Infectious Diseases 2004:39:1604-10.
30. Brown M J; Coltart J, GuneWardena K, Ritter J M, Auton T R, Glovers J F. Randomized double-blind placebo-controlled study of an angiotensin immunotherapeutic vaccine (PMD3117) in hypertensive subjects. Clinical Science 2004:107:167-73.
31. Lamb D J, Tickner M L, Dreux A C, El-Sankary W, Hourani S M, Eales-Reynolds L J, et al. Impairment of vascular function following BCG immunisation is associated with immune response to HSP-60 in cholesterol-fed rabbit. Atherosclerosis 2004:172:13-20.
32. Hill A B. The environment and disease, association or causation. Proceedings of the Royal Society of Medicine 1965:58:295-300.
33. Kautiainen S, Rimpela A, Vikat A, Viratanen S M. Secular trends in overweight and obesity among Finnish adolescents in 1977-1999. International J Obes Metab Disord 2002:26:544-52.
34. Whitlock E P, Williams S B, Gold R, Smith P R, Shipman S A. Screening and interventions for childhood overweight: a summary of evidence for the US preventive task force. Pediatrics 2005:116:e125-43.
35. Ogden C L, Flegal K M, Carroll M D, Johnson C L. Prevalence and trends in overweight among US children and adolescents, 1999-2000. JAMA 2002:288:1728-32.
36. Vanhala M J, Vanhala P T, Keinanen-Kiukaaniemi S M, Kumpusalo E A, Takala J K. Relative weight gain and obesity as a child predict metabolic syndrome as an adult. Int J Obes RelMetab 1999:23:656-59.
37. Muntner P, He J, Cutler J A, Wildman R P, Whelton P K. Trends in blood pressure among children and adolescents. JAMA 2004:291:2107-13.
38. Sorof J, Daniels S. Obesity hypertension in children, a problem of epidemic proportions. Hypertension 2002:40:441-47.
39. Doll S, Paccaud F, Bovet P, Burnier M, Wietlisbach V. Body mass index, abdomina adiposity and blood pressure: consistency of their association across developing and developed countries. International Journal of Obesity 2002:26:48-57.
40. Duncan B B, Schmidt M I. Chronic activation of the innate immune system may underlie the metabolic syndrome. Sao Paulo Medical Journal 2001:119:122-27.
41. Laaksonen D E, Niskanen L, Nyyssonen K, Punnonen K, Tuomainen T P, Valkonen V P, et al. C-reactive protein and the development of metabolic syndrome and diabetes in middle age men. Diabetologia 2004:47:1403-10.
42. Han T S, Sattar N, Williams K, Gonzalez-Villalpando C, Lean M E J, Haffner S M. Prospective study of C-reactive protein in relation to the development of diabetes and metabolic syndrome in the Mexico City Diabetes Study. Diabetes Care 2002:25:2016-21.
43. Wisse B E. The inflammatory syndrome: the role of adipose tissue cytokines in metabolic disorders linked to obesity. J Am S Nephrology 2004:15:2792-800.
44. Pradhan A D, Manson J E, Rifa N, Buring J E, Ridker P M. C-reactive protein, interleukin 6, and risk of developing type 2 diabetes mellitus. JAMA 2001:286:327-34.
45. Sesso H D, Buring J E, Rifai N, Blake G J, Gaziano J M, Ridker P M. C-reactive protein and the risk of developing hypertension. JAMA 2003:290:2945-51.
46. Vlachopoulos C, Dima I, Aznaouridis K, Vasiliadou C, Ioakeimidis N, Aggeli C, et al. Acute systemic inflammation increases arterial stiffness and decreases wave reflections in healthy individuals. Circulation 2005:112:2193-200.
47. Visser M, Bouter L M, McQuillan G M, Wener M H, Harris T B. Low-grade systemic inflammation in overweight children. Pediatrics 2001:107:e13-19.
48. Karelis A D, Faraj M, Bastard J P, St-Pierre D H, Brochu M, Prodhumme D, et al. The metabolically healthy but obese individuals presents a favorable inflammation profile. Journal of Clinical Endocrinology and Metabolism 2005:90:4145-50.
49. Bjomtorp P, Rosmond R. Hypothalmic origin of the metabolic syndrome X. Annals of the New York Academy of Sciences 1999:892:297-307.
50. Andrew R, Gale C R, Walker B R, Seck J R, Martyn C N. Glucocorticoid metabolism and the metabolic syndrome: associations in an elderly population. Exp Clinical Endocrinol Diabetes 2002:110:284-90.
51. Brunner E J, Hemingway H, Walker B R, Page M, Clarke P, Juneja M, et al. Adrenocortical, autonomic, and inflammatory causes of the metabolic syndrome: nested case control study. Circulation 2002:106:2659-65.
52. Bahr V, Pfeiffer A F, Diederich S. The metabolic syndrome X and peripheral cortisol synthesis. Exp Clinical Endocrinol Diabetes 2002:110:313-18.
53. Walker B R, Soderberg S, Lindahl B, Olsson T. Independent effects of obesity and cortisol in predicting cardiovascular risk factors in men and women. Journal of Internal Medicine 2000:247:198-204.
54. Rosmond R, Bjorntorp P. The hypothalmic-pituitary-adrenal axis activity as a predictor of cardiovascular disease, type 2 diabetes and stroke. Journal of Internal Medicine 2000:247:188-97.
55. Oltmanns K M, Dodt B, Schultes B, Raspe H H, Schweiger U, Born J, et al. Cortisol correlates with metabolic disturbances in a population study of diabetic patients. European Journal of Endocrinology 2006:154:325-31.
56. Esteve E, Ricart W, Fernadez-Real J M. Dyslipidemia and inflammation: an evolutionary conserved mechanism. Clinical Nutrition 2005:24:16-31.
57. Ershler W B, Sun W H, Binkley N, Gravenstein S, Volk M J, Kamoske G, et al. Interleukin-6 and aging: blood levels and mononuclear cell production increase with advancing age and in vitro production is modifiable by dietary restriction. Lymphokine Cytokine Res 1993:12:225-30.
58. Roubenoff R, Harris T B, Abad L W, Wilson P W, Dallal G E, Dinarello C A. Monocyte cytokine production in an elderly population: effect of age and inflammation. J Gerontol A Biol Sci Med Sci 1998:53:M20-26.
59. Rey A D, Klusman I, Besedovsky H O. Cytokines mediate protective stimulation of glucocorticoid output during autoimmunity, involvement of IL-1. American Journal Physiol Regulatory Integrative Comp Physiol 1998:275:1146-51.
60. Angeli A, Masera R G, Sartori M L, Fortunati N, Racca S, Dovio A, et al. Modulation by cytokines of glucocorticoid action. Annals of the New York Academy of Sciences 1999:876:210-20.
61. Bethin K E, Voght S K, Muglia L J. Interleukin-6 is an essential, corticotropin-releasing-hormone-independent stimulator of the adrenal axis during immune system activation. Proc Natl Acad Sci USA 2000:97:9317-22.
62. Turnbull A V, Prehar S, Kennedy A R, Little R A, Hopkins S J. Interleukin-6 is an afferent signal to the hypothalmo-pituitary-adrenal axis during local inflammation in mice. Endocrinology 2003:144:1894-906.
63. Vallieres L, Rivest S. Interleukin-6 is a needed proinflammatory cytokine in the prolonged neural activity and transcriptional activation of corticotropin-releasing factor during endotoxemia. Endocrinology 1999:140:3890-903.
64. Senn J J, Klover P J, Nowak I A, Mooney R A. Interleukin-6 induces cellular insulin resistance in hepatocytes. Diabetes 2002:51:3391-99.
65. Hall G V, Steensberg A, Sacchetti M, Fischer C, Keller C, Schjerling P, et al. Interleukin-6 stimulates lipolysis and fat oxidation in humans. Journal of Clinical Endocrinology and Metabolism 2003:88:3005-10.
66. Nonogaki K, Fuller G M, Fuentes N L, Moser A H, Staprans I, Grunfeld C, et al. Interleukin-6 stimulates hepatic triglyceride secretion in rats. Endocrinology 1995:136:2143-49.
67. Lyngso D, Simonsen L, Bulow J. Metabolic effects of interleukin-6 in splanchic and adipose tissue. Journal of Physiology 2002:543:379-86.
68. Bloomgarden Z T. Type 2 diabetes in the young, resolving epidemic. Diabetes Care 2004:27:998-1010.
69. Tuomilehto J, Karvonen M, Pitkaniemi J, Virtala E, Kohtamaki K, Toivanen L, et al. Record high incidence of type 1 (insulin dependent) diabetes mellitus in Finnish children. Diabetologia 1999:42:655-60.
70. Rowe J, Yerkovich S T, Richmond P, Suriyaarachchi D, Fisher E, Feddema L, et al. Th-2 local reactions to the acellular diphtheria-tetanus-pertussis vaccine in 4 to 6 year old children. Infection and Immunity 2005:73:8130-35.
71. Bernstein E D, Gardner E M, Abrutyn E, Gross P, Murasko D M. Cytokine production after influenza vaccination in a healthy elderly population. Vaccine 1998:16:1722-31.
72. Yousfi M E, Mercier S, Breuille D, Denis P, Papet I, Mirand P P, et al. The inflammatory response to vaccination is altered in the elderly. Mech Ageing Dev 2005:126:874-81.
73. Pourcyrous M, Korones S B, Crouse D, Bada H S. Interleukin-6, C-reactive protein, and abnormal cardiorespiratory responses to immunization in premature infants. Pediatrics 1998:101:e3-8.
74. Oken E, Kasper D L, Gleason R E, Adler G K. Tetanus toxoid stimulation of the hypothalmic-pituitary-adrenal axis correlates inversely with the increase in tetanus toxoid antibody titers. Journal of Clinical Endocrinology and Metabolism 1998:83:1691-96.
75. Levitt N S, Lambert E V, Woods D, Hales N, Andrew R, Seckl J. Impaired glucose tolerance and impaired blood pressure in low birth weight, non obese, young South African adults: early programming of the cortisol axis. Journal of Clinical Endocrinology and Metabolism 2000:85:4611-18.
76. Phillips D I W, Barker D J P, Fall C H D, Seckl J R, Whorwood C B, Wood P J, et al. Elevated plasma cortisol concentrations: a link between low birth weight and insulin resistance syndrome. Journal of Clinical Endocrinology and Metabolism 1998:83:757-60.
77. Seckl J R, Meaney M J. Glucocorticoid programming. Annals of the New York Academy of Sciences 2004:1032:63-68.
78. Boney C M, Verma A, Tucker R, Vohr B R. Metabolic syndrome in childhood: association with birth weight, maternal obesity, and gestational diabetes mellitus. Pediatrics 2005:115:290-96.
79. Strauss R S, Pollack H A. Epidemic increase in childhood overweight, 1986-1998. JAMA 2001:286:2845-48.
80. Troiano R P, Flegal K M. Overweight children and adolescents: description, epidemiology, and demographics. Pediatrics 1998:101:497-504.
81. Onkamo P, Vaananen S, Karvonen M, Tuomilehto J. Worldwide increase in the incidence of type 1 diabetes—the analysis of the data on published incidence trends. Diabetologia 1999:42:1395-403.
82. Hoshino H, Ohmori M, Uchimura K, Yamauchi Y. influences on the adoption of new tuberculosis mass-examination system for school children on tuberculosis of school age. Kekkaku 2005:80:475-79.
83. Urakami T, Kubota S, Nitadori Y, Harada K, Owada M, Kitagawa T. Annual incidence and clinical characteristics of type 2 diabetes in children as detected by urine glucose screening in the Tokyo Metropolitan Area. Diabetes Care 2005:28:1876-81.
84. Urakami T, Morimoto S, Nitadori Y, Harada K, Owada M, Kitagawa T. Recent change in the annual incidence of childhood type 2 diabetes in the Tokyo metropolitan area. Clin Pediatr Endocrinol 2007:16:53-58.
85. Urakami T, Owada M, Kitagawa T. Recent trend toward the decrease in the incidence of type 2 diabetes in Tokyo. Diabetes Care 2006:29:2176-77.
86. Lewis M, Ramsay D S, Kawakami K. Differences between Japanese infants and caucasian American infants in Behavioral and cortisol response to inoculation. Child Development 1993:64:1722-31.
87. Ogawa Y, Uchigata Y, Otani T, Iwamoto Y. Proportion of diabetes type in Early-Onset Diabetes in Japan. Diabetes Care 2007:30:e30.
88. Dabela D. Incidence of diabetes in youth in the United States. JAMA 2007:297:2716-24.
89. Pinhas-Hamiel O, Zeitler P. The global spread of type 2 diabetes mellitus in children and adolescents. J Pediatrics 2005:146:693-700.
90. Campbell-Stokes P L, Taylor B J. Prospective incidence study of diabetes mellitus in New Zealand children age 0-14. Diabetologia 2005:48:643-48.
91. Craig M E, Femia G, Broyda V, Lloyd M, Howard N J. Type 2 diabetes in indigenous and non-indigenous children and adolescents in New South Wales. The Medical Journal of Australia 2007:186:497-99.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference. Reference to steps or compositions as being_conventional_, _standard_, _usual_, _known_ or the like is not to be considered an admission that any aspect, description or embodiment of the present invention is disclosed, taught or suggested in the relevant art.

Any description of an embodiment as_desirable_or_preferable_ is intended to imply that the invention is not limited to the stated embodiment, but rather covers alternatives whether mentioned or not. Any description of a class or range as being useful or preferred in the practice of the invention shall be deemed a description of any subclass or subrange contained therein, as well as a separate description of each individual member or value in said class or range.

TABLE 1

| | | | | | Diastolic | % Obese | % Obese | % Obese |
|---|---|---|---|---|---|---|---|---|
| YEAR | VALENCE | DOSES | VACCINES | MS | BP | age 12 | age 6 | age 2 |
| 1978 | 32 | 24 | 7 | | | 4.8 | 6 | 5 |
| 1979 | 32 | 24 | 7 | | | | | |
| 1980 | 32 | 24 | 7 | | | | | |
| 1981 | 32 | 24 | 7 | | | | | |
| 1982 | 32 | 24 | 7 | | | | | |
| 1983 | 32 | 24 | 7 | | | | | |
| 1984 | 32 | 24 | 7 | | | | | |
| 1985 | 32 | 24 | 7 | | | | | |
| 1986 | 32 | 24 | 7 | | | | | |
| 1987 | 32 | 24 | 7 | | | | | |
| 1988 | 32 | 24 | 7 | | | | | |
| 1989 | 35 | 27 | 7 | | | | | |
| 1990 | 35 | 27 | 7 | | | | | |
| 1991 | 38 | 30 | 8 | 4.2 | 58.4 | 10.6 | 10.6 | 7.2 |
| 1992 | 42 | 34 | 9 | | | | | |
| 1993 | 42 | 34 | 9 | | | | | |
| 1994 | 42 | 34 | 9 | | | | | |
| 1995 | 42 | 34 | 9 | | | | | |
| 1996 | 43 | 35 | 10 | | | | | |
| 1997 | 43 | 35 | 10 | | | | | |
| 1998 | 43 | 35 | 10 | | | | | |
| 1999 | 47 | 36 | 11 | 6.4 | 61.7 | 15.5 | 15.3 | 10.4 |
| 2000 | 71 | 39 | 11 | | | | | |
| 2001 | 71 | 39 | 11 | | | | | |
| 2002 | 71 | 39 | 11 | | | | | |
| 2003 | 77 | 41 | 11 | | | | | |
| 2004 | 77 | 41 | 11 | | | | | |
| 2005 | 77 | 41 | 11 | | | | | |
| 2006 | 84 | 45 | 13 | | | | | |
| | | | | | | DOSES | DOSES | DOSES |
| Doses | P | | | | | 0.031 | 0.004 | 0.05 |
| | CORRELATION | | | | | 0.9988 | 1 | 0.9943 |
| | | | | | | VALENCE | VALENCE | VALENCE |
| Valence | P | | | | | 0.104 | 0.069 | 0.005 |
| | CORRELATION | | | | | 0.9867 | 0.9941 | 1 |
| | | | | | | VACCINES | VACCINES | VACCINES |
| Vaccine | p | | | | | 0.21 | 0.175 | 0.111 |
| | CORRELATION | | | | | 0.9462 | 0.9625 | 0.9848 |

P value
P < 0.001
P < 0.001

TABLE 2
Rates of Type 2 Diabetes in BCG Vaccinated and Unvaccinated Children

| | | Elementary School | | | Junior High School | | |
|---|---|---|---|---|---|---|---|
| | | | Cases | Incidence | | Cases | Incidence |
| | Year | Population | Type 2 DM | per 100,000 | Population | Type 2 DM | per 100,000 |
| BCG Vaccinated | 1982 | 254,697 | 3 | | 126,811 | 10 | |
| | 1983 | 241,793 | 2 | | 125,427 | 8 | |
| | 1984 | 228,851 | 1 | | 123,893 | 10 | |
| | 1985 | 214,655 | 3 | | 125,404 | 10 | |
| | 1986 | 210,563 | 1 | | 129,061 | 12 | |
| | 1987 | 213,617 | 0 | | 131,667 | 7 | |
| | 1988 | 205,669 | 4 | | 122,731 | 7 | |
| | 1989 | 204,940 | 1 | | 114,777 | 5 | |
| | 1990 | 197,725 | 2 | | 106,269 | 11 | |
| | 1991 | 210,832 | 0 | | 108,625 | 4 | |
| | 1992 | 204,306 | 2 | | 103,549 | 6 | |
| | 1993 | 198,283 | 2 | | 96,766 | 10 | |
| | 1994 | 192,697 | 2 | | 91,771 | 7 | |
| | 1995 | 186,653 | 2 | | 88,079 | 8 | |
| | 1996 | 188,782 | 2 | | 90,057 | 2 | |
| | 1997 | 178,134 | 2 | | 85,794 | 7 | |
| | 1998 | 174,119 | 4 | | 83,345 | 5 | |
| | 1999 | 170,539 | 3 | | 79,893 | 4 | |
| | 2000 | 168,625 | 2 | | 77,268 | 4 | |
| | 2001 | 172,505 | 1 | | 76,950 | 3 | |
| | 2002 | 169,706 | 1 | | 73,227 | 4 | |
| | Totals | 4,187,691 | 40 | 0.96 | 2,161,364 | 144 | 6.66 |
| Not Vaccinated | 2003-2004 | 307,213 | 1 | 0.33 | 122,690 | 3 | 2.45 |
| | | | | RR = 2.93 | | | RR = 2.72 |

Two Strata Wighted Relative Risk: 2.78
1.03 < RR < 7.48

TABLE 3
Incidence of Type 1 Diabetes versus Type 2 Diabetes by Race

| Age 10-19 | Yearly Incidence/100,000 | | Age 0-14 | Yearly Incidence/100,000 | |
|---|---|---|---|---|---|
| Race | Type 1 | Type 2 | Race | Type 1 | Type 2 |
| Indians | 5.94 | 36.96 | Indians | 6.1 | 9.39 |
| Asians | 7.44 | 17.1 | Asians | 7.37 | 4.8 |
| Hispanics | 14.98 | 12.79 | Hispanics | 14.11 | 3.39 |
| Blacks | 15.34 | 20.98 | Blacks | 15.18 | 8.61 |
| US Whites | 24.1 | 4.25 | Whites | 26.79 | 1.16 |
| Aboriginals | 15.1 | 12.7 | Maori | 5.6 | 1.78 |
| Australian Whites | 21.4 | 2.1 | Non Maori | 21.7 | 0.39 |

correlation coeficient −0.85
P < 0.05
correlation coefficinet −0.5
P < 0.05

1. An method for safely immunizing a human with one or more doses of one or more immunogens which induce production of antibodies or activation of T cells in said human, by administering one or more doses of one or more said immunogens, thereby inducing the production of antibodies or activation of T cells in said human and thus conferring protective immunity to one or more infectious diseases when administered according to one or more immunization schedules, said method comprising
(I) assessing the risk of metabolic syndrome and or one of its component diseases/disorders which is associated with said immunization schedule or said one or more immunogens,
(II) screening one or more potential recipients and identifying at least one human subject who would be expected to be immunized safely with said one or more immunogens or said immunization schedule reflective of the analysis from (I), and
(III) immunizing said human against said one or more infectious diseases.

2. The method of claim 1 in which the risk of at least two component diseases/disorders of metabolic syndrome is considered.

3. The method of claim 1 where data on at least two immunization schedules is assessed.

4. The method of claim 3 where said human is immunized in III according to a schedule which is associated with a lower risk compared to another schedule.

5. The method of claim 3 wherein one schedule provides at least one dose of at least one immunogen not provided by another schedule.

6. The method of claim 3 wherein said at least one schedule contains at least one more dose of at least one immunogen than another schedule.

7. The method of claim 3 wherein said at least one schedule contains at least one larger total dose of at least one immunogen than another schedule.

8. The method of claim 3 wherein at least one schedule contains at least one more valence than another schedule.

9. The method of claim 1 where the risk is assessed at a time frame of at least 6 months after said one or more immunogens is first administered.

10. The method of claim 1 where the risk is assessed at a time frame of at least 1 year after said one or more immunogens is first administered.

11. The method according to claim 1 wherein said assessing the risk of metabolic syndrome and or one of its components associated with said immunization schedule or said one or more immunogens comprises assessing the effect of race, family history, or cortisol activity on said risk of metabolic syndrome and or one of its components.

12. The method of claim 1 wherein assessing the risk of metabolic syndrome and or one of its component diseases/disorders comprises assessing the risk of obesity.

13. The method of claim 1 wherein assessing the risk of metabolic syndrome and or one of its component diseases/disorders comprises assessing the risk hypertension.

14. The method of claim 1 wherein assessing the risk of metabolic syndrome and or one of its component diseases/disorders comprises assessing the risk hypertriglyceridemia or hyperlipidemia.

15. The method of claim 1 wherein at least one component disease/disorder of metabolic syndrome comprises assessing the risk insulin resistance.

16. The method of claim 1 wherein at least one component disease/disorder of metabolic syndrome comprises assessing the risk microalbuminurea.

17. The method of claim 1 wherein at least one component disease/disorder of metabolic syndrome comprises assessing the risk low HDL cholesterol.

18. The method of claim 1 where at least one immunogen is from the group consisting of a measles, mumps, rubella, diphtheria, pertussis, hemophilus influenza, tetanus, hepatitis A, hepatitis B, varicella, influenza, pneumococcus, meningococcus, rotavirus and polio immunogens.

19. The method of claim 1 where at least one immunogen is a non-pediatric immunogen from the group consisting of anthrax, plague, encephalitis, Hepatitis C, hepatitis e, typhus, typhoid fever, streptococcus, staphylococcus, lyme disease, cholera, campylobacter, helicobacter, E. coli, shigella, leishmania, leprosy, cytomegalovirus (CMV), human papilloma virus, respiratory syncytial virus, Epstein Barr virus, herpes, parainfluenza, adenovirus, human immunodeficiency virus (HIV), hepatitis A, NonA NonB hepatitis, rabies, yellow fever, rabies, Japanese encephalitis, flavivirus, dengue, west nile virus, avian flu virus, SARS coronaviruses, toxoplasmosis, coccidiomycosis, schistosomiasis, and malaria immunogens.

20. The method of claim 1 where assessing the risk comprises data on risk in a group of at least 200 people.

21. A method for more safely marketing one or more doses of one or more immunogens which are protective against one or more infectious diseases when administered according to one or more immunization schedules, said method comprising providing information, or assisting in the dissemination of information, on the risk of developing metabolic syndrome and or one of its component diseases/disorders, and marketing one or more of said immunogens for use in one or more immunization schedules.

22. The method of claim 21 wherein said information helps determine who to immunize, when to immunize, or with what to administer.

23. The method of claim 21 wherein said information helps determine what schedule to administer.

24. A method for safely immunizing a human with one or more doses of one or more immunogens which induce protective immunity to one or more cancers when administered according to one or more immunization schedules, said method comprising
(I) assessing the risk of said metabolic syndrome and or one of its components associated with said immunization schedule or said one or more immunogens
(II) screening one or more potential recipients and identifying at least one human subject who would be expected to be immunized safely with said one or more immunogens or said immunization schedule reflective of the analysis from (I), and
(III) immunizing said human against said one or more cancers.

25. A method for safely administering to a human with one or more doses of one or more immune modulators which induce the production or activation of an immune system component and thereby confer protective immunity to one or more diseases when administered according to one or more schedules, said method comprising
(I) assessing the risk of said metabolic syndrome and or one of its components associated with said schedule or said one or more immune modulators
(II) screening one or more potential recipients and identifying at least one human subject who would be expected to be safely receive said one or more immune modulators reflective of the analysis from (I), and
(III) administering to a human said one or more immune modulators which induce the production or activation of an immune system component in said human.

26. (canceled)

27. The method of claim 1, wherein said immunizing comprises injection of at least one dose of at least one immunogen by means of an injection device.

28. The method of claim 1, wherein said assessing of risk comprises analysis of statistics by means of a computer programmed with and running a statistics program.

29. The method of claim 1, wherein said immunizing comprises administration of an immunogen by means of a depot adjuvant which releases at least one immunogen in vivo over a prolonged, period.

30. The method of claim 1 in which at least one immunogen is a recombinant hepatitis B immunogen.

31. The method of claim 1 in which at least one immunogen is an acellular pertussis immunogen.

32. The method of claim 1 which further comprises providing a kit which comprises at least one receptacle comprising one or more doses of at least one immunogen, and labeling comprising directions for use and warnings concerning possible adverse effects.

33. The method of claim 1 wherein said screening of potential recipients comprises screening the potential recipients for a family medical history or personal medical history of metabolic syndrome, hypertension, obesity, hypertriglyceridemia, hyperlipidemia, low HDL cholesterol, insulin resistance or microalbuminurea.

34. The method of claim 33 which comprises providing a data storage device, said device comprising a data structure comprising medical records for potential recipients, said records comprising fields providing said family history or personal history.

35. The method of claim 1 wherein risk information is disseminated electronically.
